(19) United States
(12) Patent Application Publication
(10) Pub. No.: US 2016/0341499 A1
(43) Pub. Date: Nov. 24, 2016

(54) PROCESS FOR ONLINE CLEANING OF MTO REACTOR EFFLUENT COOLER

(71) Applicant: UOP LLC, Des Plaines, IL (US)

(72) Inventor: Daniel A. Kauff, Arlington Heights, IL (US)

(21) Appl. No.: 14/715,898

(22) Filed: May 19, 2015

(51) Int. Cl. F28G 9/00; C07C 1/20

(52) U.S. Cl. CPC F28G 9/00; C07C 1/20

(57) ABSTRACT

A process is presented for the online cleaning of the tubes in a heat exchanger. The process includes adding an abrasive material to the gas stream to be cooled, and abrading any particles or catalyst fines that have adhered to the tube walls. The abrasive material is to be a water soluble solid to allow for the removal of the abrasive material in a quench tower.

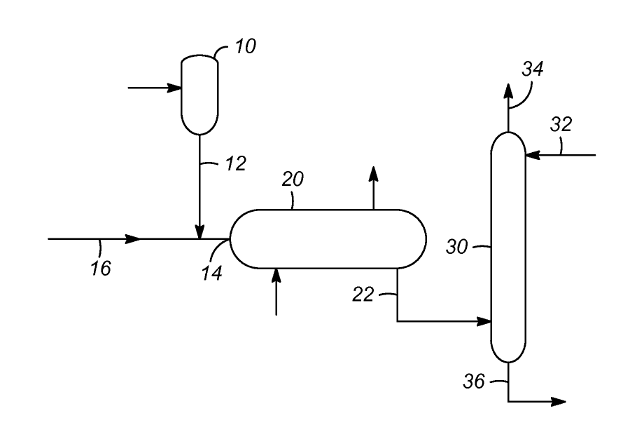

### FIELD OF THE INVENTION

The present invention relates to the cleaning of cooling units. In particular, this invention relates to the cleaning of heat exchangers wherein the surfaces can be fouled by a buildup of particulate matter.

### BACKGROUND

Heat exchangers are a basic unit in the petrochemical processing industry. Heat exchangers are used in Fluid Catalytic Cracking (FCC) units and Methanol to Olefins (MTO) units for cooling flue gases to recover heat, and the flue gases often contain small particles. The small particles are often catalyst fines that pass through a system and adhere to the heat exchanger tubes. Periodically the heat exchanger tubes need to be cleaned. In order to continue the use of the heat exchangers, the heat exchangers are cleaned online. In FCC and MTO units, flue gas coolers are cleaned by the injection into a flue gas stream of abrasive particles. A typical abrasive can be a hard material such as walnut shells that have been broken up to sizes in the range of 1.7 to 3.5 mm. Walnut shells are organic materials, and they burn completely before exiting the flue gas stack.

Fouling of coolers reduces the heat exchange efficiencies, and can restrict flows, which changes the contact times for the gas streams to be cooled. These loss of efficiencies can be costly and change the overall operating conditions of downstream processing units.

However, not all flue gas coolers have the same conditions, and therefore, a typical means of cleaning FCC and MTO flue gas coolers is inappropriate for cleaning other types of effluent gas coolers. MTO reactor effluent coolers are not ideal for using the same materials as are used in FCC and MTO flue gas coolers. Therefore, different materials or means of cleaning MTO reactor coolers is needed.

### SUMMARY

The present invention is a process for online cleaning of heat exchanger tubes.

A first embodiment of the invention is a process for cleaning effluent coolers comprising passing abrasive pellets to a blowpot; pressuring the blowpot; passing the pellets in the blowpot through a transfer tube to the tube side of a heat exchanger; mixing a reactor effluent gas with the pellets; and passing the pellets through the tubes of the heat exchanger, thereby removing particle buildup in the tube side of the heat exchanger. An embodiment of the invention is one, any or all of prior embodiments in this paragraph up through the first embodiment in this paragraph further comprising generating a cooler effluent stream comprising cooled effluent gas and abrasive pellets. An embodiment of the invention is one, any or all of prior embodiments in this paragraph up through the first embodiment in this paragraph further comprising passing the cooler effluent stream to a quench tower to generate a cooled and quenched effluent stream with a reduced abrasive pellet concentration. An embodiment of the invention is one, any or all of prior embodiments in this paragraph up through the first embodiment in this paragraph wherein the quench tower generates a liquid quench stream that comprises cooling water and dissolved abrasive pellets. An embodiment of the invention is one, any or all of prior embodiments in this paragraph up through the first embodiment in this paragraph further comprising passing the liquid quench stream to a water recovery unit to generate a water stream and a waste water stream; and recycling the water stream to the quench tower. An embodiment of the invention is one, any or all of prior embodiments in this paragraph up through the first embodiment in this paragraph wherein the abrasive pellets comprise calcium hydroxide. An embodiment of the invention is one, any or all of prior embodiments in this paragraph up through the first embodiment in this paragraph wherein the abrasive pellets are soluble in water. An embodiment of the invention is one, any or all of prior embodiments in this paragraph up through the first embodiment in this paragraph wherein the abrasive pellets are sized between 1 and 4 mm. An embodiment of the invention is one, any or all of prior embodiments in this paragraph up through the first embodiment in this paragraph wherein the abrasive pellets are sized between 2 and 3 mm.

A second embodiment of the invention is a process for cleaning cooling tubes comprising passing a hot effluent gas to a heat exchanger; passing an abrasive material to the heat exchanger, thereby generating an abrasive-effluent gas mixture; and passing the abrasive-effluent gas mixture to a quench tower, thereby generating a quenched effluent gas, and an aqueous stream comprising the abrasive material. An embodiment of the invention is one, any or all of prior embodiments in this paragraph up through the second embodiment in this paragraph wherein the abrasive material is soluble in water. An embodiment of the invention is one, any or all of prior embodiments in this paragraph up through the second embodiment in this paragraph wherein the abrasive material comprises calcium hydroxide. An embodiment of the invention is one, any or all of prior embodiments in this paragraph up through the second embodiment in this paragraph wherein the abrasive material comprises pellets that are sized between 1 and 4 mm. An embodiment of the invention is one, any or all of prior embodiments in this paragraph up through the second embodiment in this paragraph wherein the abrasive pellets are sized between 2 and 3 mm. An embodiment of the invention is one, any or all of prior embodiments in this paragraph up through the second embodiment in this paragraph further comprising passing an abrasive material to a blowpot; pressuring the blowpot; and passing the abrasive material in the blowpot through a transfer tube to the tube side of a heat exchanger.

A third embodiment of the invention is a process for cleaning effluent coolers comprising passing an abrasive material to a blowpot; pressuring the blowpot; passing the abrasive material in the blowpot through a transfer tube to the tube side of a heat exchanger; passing a hot effluent gas to the heat exchanger; mixing the hot effluent gas with the abrasive material to generate a mixture; and passing the mixture through the tubes of the heat exchanger, thereby removing particle buildup in the tube side of the heat exchanger, and generating a heat exchanger effluent. An embodiment of the invention is one, any or all of prior embodiments in this paragraph up through the third embodiment in this paragraph wherein the abrasive material comprises pellets that are sized between 1 and 4 mm An embodiment of the invention is one, any or all of prior embodiments in this paragraph up through the third embodiment in this paragraph further comprising passing the heat exchanger effluent to a quench tower, thereby generating a quenched effluent gas, and an aqueous stream comprising the abrasive material. An embodiment of the invention is one, any or all of prior embodiments in this paragraph up through the third embodiment in this paragraph wherein the abrasive material comprises a water soluble material. An embodiment of the invention is one, any or all of prior embodiments in this paragraph up through the third embodiment in this paragraph wherein the abrasive material comprises calcium hydroxide.

Other objects, advantages and applications of the present invention will become apparent to those skilled in the art from the following detailed description and drawings.

### BRIEF DESCRIPTION OF THE DRAWING

The FIGURE is a configuration for online cleaning of tubes in a tube and shell heat exchanger used in a methanol to olefins conversion process.

### DETAILED DESCRIPTION

Reactor effluent coolers have a tendency to foul because small catalyst particles from the reactor are carried into the effluent coolers and adhere to the tube walls. MTO reactor effluent coolers are similar to FCC and MTO flue gas coolers, but are operated at lower temperatures, have different stream compositions, and the cooled effluent has a different process destination than FCC flue gas coolers. The use of abrasive materials used in flue gas coolers, such as walnut shells, would not be combusted or consumed and therefore would survive passing through the cooler, and enter into downstream units, such as the quench tower. The large particles of abrasive entering the quench tower can cause plugging problems in quench tower internals and in particular in nozzles and orifices.

For an MTO reactor, fresh catalyst was tried as an abrasive material, wherein the fresh catalyst was injected into the cooler, and then recovered from a quench tower bottoms stream. However, the size of fresh catalyst particles are small and were found to be ineffective due to the small particle size.

In an MTO reactor system, the reactor effluent gas passes through a heat exchanger, and is then quenched in a quench tower. The heat exchanger is a shell and tube heat exchanger with the reactor effluent gas passing through the tube side of the exchanger. The quench tower has a large water circulation loop, and a water soluble solid could be an effective abrasive material for the reactor effluent heat exchanger. A particular water soluble solid is calcium hydroxide, or lime, and is a material that is readily available and inexpensive. Another possible material is potassium hydroxide, and various other salts can also serve as an abrasive material. However, selection should limit those salts without corrosive properties for downstream equipment and salts that are not poisons to the MTO catalyst.

The present invention is a process for online cleaning of a reactor effluent cooler in an MTO process. The process includes passing abrasive pellets to a blowpot. A blowpot is a system for transferring a dense phase material, often a solid, into a flowing system. The blowpot is pressurized, and then the abrasive pellets are passed through a transfer tube from the blowpot to upstream side of a heat exchanger. In particular, a tube and shell heat exchanger, wherein the abrasive pellets are transferred upstream of the tube side of the heat exchanger and pass through the tubes. The reactor effluent gas mixes with the abrasive pellets, and the mixtures comprising the pellets is passed through the tubes of the heat exchanger. This generates a cooled effluent stream comprising the cooled effluent gas, small particles removed from the cooler and abrasive pellets.

The cooled effluent stream is passed to a quench tower where the effluent stream is contacted with a circulating water stream. The water further cools the effluent and removes solids and water soluble materials in the effluent stream. The water removes the solid pellets and generates an effluent stream with the abrasive pellets removed. The abrasive pellets are made of a water soluble solid, or salt, and are dissolved in the quench stream to generate a liquid quench stream comprising water and the dissolved abrasive pellets.

The process can further include passing the liquid quench stream to a water recovery unit to generate a water stream and a waste water stream, and recycling the water stream to the quench tower. The abrasive pellets are comprised of a water soluble solid, such as salt. A preferred material for the abrasive pellets is calcium hydroxide. The pellets need to be large enough to perform the abrasion on the tube walls, but not too large so as to plug the tubes. A preferred pellet size range is from 1 to 4 mm along the longest dimension, with a more preferred range from 2 to 3 mm.

The process as can be seen in the FIGURE, includes charging a blowpot 10 with an abrasive material. The blowpot 10 is pressurized, and the abrasive material is passed through a transfer tube 12 to the inlet 14 of the tube side of a heat exchanger 20. A hot effluent gas 16 from a reactor is passed to the heat exchanger 20. The blowpot 10 is pressurized to a pressure above the pressure of the hot effluent gas leaving the reactor. In the inlet to the heat exchanger the hot effluent gas and the abrasive material generates a mixture to be passed through the heat exchanger 20. An exchanger effluent stream 22, comprising the cooled gas, abrasive material and particles abraded from the heat exchanger tubes is passed to a quench tower 30.

A water stream 32 entering the quench tower 30 contacts the exchanger effluent stream 22 to generate a quenched effluent gas stream 34 with the solid particles and abrasive material removed. The quench tower 30 also generates an aqueous stream 36 comprising the abrasive material and the solid particles that were abraded from the heat exchanger tubes.

The aqueous stream 36 can be processed to recover water for reuse. A concentrated waste stream can be treated to either recover or dispose of catalyst fines, and to recover or dispose of the dissolved abrasive material.

While the invention has been described with what are presently considered the preferred embodiments, it is to be understood that the invention is not limited to the disclosed embodiments, but it is intended to cover various modifications and equivalent arrangements included within the scope of the appended claims.

What is claimed is:

1. A process for cleaning effluent coolers comprising:
passing abrasive pellets to a blowpot;
pressuring the blowpot;
passing the pellets in the blowpot through a transfer tube to the tube side of a heat exchanger;
mixing a reactor effluent gas with the pellets; and
passing the pellets through the tubes of the heat exchanger, thereby removing particle buildup in the tube side of the heat exchanger.

2. The process of claim 1 further comprising generating a cooler effluent stream comprising cooled effluent gas and abrasive pellets.

3. The process of claim 2 further comprising passing the cooler effluent stream to a quench tower to generate a cooled and quenched effluent stream with a reduced abrasive pellet concentration.

4. The process of claim 3 wherein the quench tower generates a liquid quench stream that comprises cooling water and dissolved abrasive pellets.

5. The process of claim 4 further comprising:
passing the liquid quench stream to a water recovery unit to generate a water stream and a waste water stream; and
recycling the water stream to the quench tower.

6. The process of claim 1 wherein the abrasive pellets comprise calcium hydroxide.

7. The process of claim 1 wherein the abrasive pellets are soluble in water.

8. The process of claim 1 wherein the abrasive pellets are sized between 1 and 4 mm.

9. The process of claim 8 wherein the abrasive pellets are sized between 2 and 3 mm.

10. A process for cleaning cooling tubes comprising:
passing a hot effluent gas to a heat exchanger;
passing an abrasive material to the heat exchanger, thereby generating an abrasive-effluent gas mixture; and
passing the abrasive-effluent gas mixture to a quench tower, thereby generating a quenched effluent gas, and an aqueous stream comprising the abrasive material.

11. The process of claim 10 wherein the abrasive material is soluble in water.

12. The process of claim 11 wherein the abrasive material comprises calcium hydroxide.

13. The process of claim 10 wherein the abrasive material comprises pellets that are sized between 1 and 4 mm.

14. The process of claim 13 wherein the abrasive pellets are sized between 2 and 3 mm.

15. The process of claim 10 further comprising:
passing an abrasive material to a blowpot;
pressuring the blowpot; and
passing the abrasive material in the blowpot through a transfer tube to the tube side of a heat exchanger.

16. A process for cleaning effluent coolers comprising:
passing an abrasive material to a blowpot;
pressuring the blowpot;
passing the abrasive material in the blowpot through a transfer tube to the tube side of a heat exchanger;
passing a hot effluent gas to the heat exchanger;
mixing the hot effluent gas with the abrasive material to generate a mixture; and
passing the mixture through the tubes of the heat exchanger, thereby removing particle buildup in the tube side of the heat exchanger, and generating a heat exchanger effluent.

17. The process of claim 16 wherein the abrasive material comprises pellets that are sized between 1 and 4 mm

18. The process of claim 16 further comprising passing the heat exchanger effluent to a quench tower, thereby generating a quenched effluent gas, and an aqueous stream comprising the abrasive material.

19. The process of claim 18 wherein the abrasive material comprises a water soluble material.

20. The process of claim 19 wherein the abrasive material comprises calcium hydroxide.
